(19) United States
(12) Patent Application Publication
(10) Pub. No.: US 2020/0188412 A1
(43) Pub. Date: Jun. 18, 2020

(54) ACTIVE ESTER DERIVATIVES OF TESTOSTERONE, COMPOSITIONS AND USES THEREOF

(71) Applicant: Acerus Biopharma Inc., Mississauga (CA)

(72) Inventors: Nathan Bryson, Toronto (CA); Avinash Chander Sharma, Brampton (CA)

(21) Appl. No.: 16/713,799

(22) Filed: Dec. 13, 2019

Related U.S. Application Data
(60) Provisional application No. 62/779,854, filed on Dec. 14, 2018.

(51) Int. Cl. A61K 31/568; A61K 9/00; A61K 47/44; A61P 35/00; A61P 15/00

(52) U.S. Cl. CPC A61K 31/568; A61K 9/0043; A61P 15/00; A61P 35/00; A61K 47/44

(57) ABSTRACT

The present invention relates to new compounds and compositions comprising active ingredient derivatives of testosterone, and novel testosterone derivatives, novel testosterone methods, novel testosterone compositions, novel testosterone articles of manufacture of pharmaceutical preparations and novel testosterone therapeutic uses thereof.32

### CROSS REFERENCE TO RELATED APPLICATION

This application claims priority to U.S. Provisional Application Ser. No. 62/779,854 filed Dec. 14, 2018, the contents of which are herein incorporated by reference.

### FIELD OF THE INVENTION

The present invention relates to new compositions comprising active ester derivatives of testosterone, methods, compositions, and pharmaceutical preparations and therapeutic uses thereof.

### BACKGROUND OF THE INVENTION

Testosterone is an endogenous sex steroid. Testosterone is the primary male sex hormone and an anabolic steroid. In male humans, testosterone plays a key role in the development of male reproductive tissues such as testes and prostate, as well as promoting secondary sexual characteristics such as increased muscle and bone mass, and the growth of body hair. In addition, testosterone is involved in health and well-being, and the prevention of osteoporosis. Insufficient levels of testosterone in children can lead to inadequate physical and sexual development, whereas in adult men low testosterone may lead to physical symptoms of low energy and low libido, in addition to abnormalities including frailty and bone loss.

Testosterone can be made into a variety of pharmaceutical preparations. In most preparations, testosterone is dissolved in the vehicle and the vehicle is applied to a portion of the body from whence the delivery occurs. For example, testosterone can be administered intranasally via a nasal pharmaceutical composition formulated with a testosterone prodrug, such as testosterone propionate, testosterone enanthate, testosterone cypionate and undecanoate. Injectable forms of testosterone and prodrug esters of testosterone are dissolved in vegetable oil or benzyl benzoate prior to injection. Topical forms have testosterone contained in alcohol or a greasy material which is applied to the skin.

Regarding the nasal route of administration, WO/2012/156820 describes a testosterone formulation wherein the active is dissolved in castor oil at a concentration of 4.5%.

Equally, U.S. patent application Ser. No. 15/507,246 describes a nasal formulation of testosterone propionate wherein the active is dissolved in a mixture of medium chain triglycerides, ethoxylated castor oil and water. Examples are provided for compositions comprising 1.2% and 2.2% testosterone for treatment of anxiety and fear.

Nasal delivery platform has several advantages. The pharmacokinetics of the drug show fast absorption (Tₘₐₓ about 45-60 min) and a fast washout after each dose.[ See Rogol et al.: Andrology, 2016, 4, 46. This particular profile appears to demonstrate a baseline endogenous testosterone level that remains constant and unchanged even after 1 year of treatment, implying that the underlying hypogonadal feedback mechanisms remain active. This further substantiated by measurements that show that the gonadotropins, luteinizing hormone and follicle stimulating hormone both remain in the normal range of values. Also, hematocrit levels are only very modestly changed when testosterone replacement is performed with nasal testosterone. Lastly, the total daily dose with nasal delivery is between 22 and 33 mg, which is substantially less than other transdermal (topical) gel products which have starting doses in the range of 50 mg and are typically administered at doses in the range Of 60-80 mg, on average. A lower dose may likely result in a better safety profile for the testosterone as there are fewer metabolites which can interact with the body's physiology and must be eliminated.

Thus, it would be advantageous to have a testosterone replacement product that could achieve all the positive aspects of a nasal testosterone, but could be useful with the application of a single dose. As the volume that can be applied in the nose with a single dose is also limited to approximately 150 uL, and therefore the aforementioned total daily dose must be contained in this volume of vehicle comprising active, which to date, has not been possible.

Thus, the present invention describes high concentration nasal delivery formulations and products. More specifically, the present invention provides new nasal formulations based on testosterone ester prodrugs.

### SUMMARY OF THE INVENTION

The present invention overcomes the disadvantageous and drawbacks of the prior art nasal testosterone formulations through the discovery of high concentration nasal delivery testosterone formulations and products. More specifically, the present invention provides new nasal formulations based on testosterone ester prodrugs. In accordance with the present invention, esters of testosterone, such as propionate, enanthate, cypionate and undecanoate, show greatly improved solubility in vegetable oils.

### DETAILED DESCRIPTION AND PREFERRED EMBODIMENTS

In accordance with the present invention and as used herein, the following terms are defined with the following meaning, unless explicitly stated otherwise.

A “testosterone ester” as used in the context of this application is a derivative of testosterone comprising at least a substitution on the hydroxyl group on the cyclopentyl ring of the steroid core with an acyl functional group or a substituted acyl functional group as those functional groups are defined below. When a carbon limit is assigned to a testosterone ester, the carbon limit is relative only to the carbon atoms on the acyl substitution. The terms “testosterone ester”, “testosterone derivative” or “testosterone prodrug” are used interchangeably unless specifically noted otherwise, and are distinct from the parent molecule “testosterone”.

The term “physiologically cleavable ester” refers to a derivative of the hydroxyl of formula (I) and an acid or acid derivative, wherein the product is cleaved in the body to give the compound formula (I) or an active metabolite. Such a physiologically cleavable ester can be viewed as a “pro-drug.” Such a “pro-drug” is valuable if it increases the bioavailability of the corresponding hydroxyl compound when such a pro-drug is administered to a subject. For example, a “pro-drug” administered intranasally may be more readily absorbed into the blood, may facilitate the delivery of the parent compound to a biological compartment of the subject such as the brain or lymphatic, which may also have more favorable patient acceptance, safety profiles and/or pharmacokinetics for specific tailoring to subjects for use in the intended indication. A general overview of pro-drugs is provided in (1) “Pro-drugs As Novel Delivery Systems,” Vol. 14 of the ACS Symposium Series, by T. Higuchi and V. Stella, and (2) “Bioreversible Carriers in Drug Design,” American Pharmaceutical Association, Porgamon Press, 1987, Edward B. Roche, Ed.

Formula 1 in accordance with the present inventions is as follows:

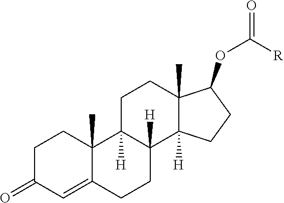

Testosterone is esterified in various pharmaceutical preparations, with esters of propionate, enanthate, cypionate and undecanoate being marketed as oral or injectable formulations for the treatment of hypogonadism.

Carboxylic acids that form the “carbonyl group” of the ester, i.e., —C(O)—R, that can be used as derivatives according to the invention and form the “pro-drug” include mono-carboxylic acids that are derived from unsubstituted or substituted lower linear or branched chain alkyl, alkenyl, alkynyl or arylakyl entities. R is defined for example in paragraphs [0020]-[0036]. Naturally occurring carboxylic acids are generally a preferred class of that may as acceptable, cleavable esters of a pharmaceutically-active ingredient.

The term “lower alkyl” carboxylic acid refers to a monovalent, saturated aliphatic hydrocarbon radical having from one to twelve (12) carbon atoms bonded to a carboxyl group. Alkyl may be a straight chain (i.e. linear), a branched chain, or a cyclic structure. Representative examples of lower alkyl radicals include methyl, ethyl, n-propyl, n-butyl, n-pentyl, n-hexyl, isopropyl, isobutyl, isopentyl, amyl, sec-butyl, tert-butyl, tert-pentyl, cyclopropyl, cyclobutyl, cyclopentylethyl (cypionate), undecanoate and the like.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃₃ (tert-butyl, t-butyl, t-Bu or ^{t}Bu), and —CH₂C(CH₃₃ (neo-pentyl) are non-limiting examples of alkyl groups.

The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH3, —OCH₂CH₃, ——C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl.

The term “alkenyl” when used without the “substituted” modifier refers to an monovalent unsaturated aliphatic group with a carbon atom as the point of attachment, a linear or branched acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂.

The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH.dbd.C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —CCH, —CCCH₃, and —CH₂CCCH₃ are non-limiting examples of alkynyl groups. An “alkyne” refers to the compound H—R, wherein R is alkynyl.

When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —HCH₂CH₃, —N(CH₃)₂, —C(O)NH₂, —OC(O)CH₃, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

An “arene” refers to the compound H—R, wherein R is aryl as that term is defined above. Benzene and toluene are non-limiting examples of arenes.

When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “aralkyl” when used without the “substituted” modifier refers to the monovalent group—alkanediyl—aryl, in which the terms alkanediyl and aryl are each used in a manner consistent with the definitions provided above. Non-limiting examples are: phenylmethyl (benzyl, Bn) and 2-phenyl-ethyl.

When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, ——C(O)CH₃, —NHCH₃, —NHCH₂CH₃, —N(CH₃)₂, —C(O)NH₂, —OC(O)CH₃, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, C(O)CH(CH₂)₂, C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —SH, —OCH₃, —OCH₂CH₃, —NHCH₃, —NHCH₂CH₃, —N(CH₃)₂, —OC(O)CH₃, or —S(O)₂NH₂. The groups, —C(O)CH₂CF₃, —CO₂ (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The term lower “alkenyl” carboxylic acid refers to an aliphatic group that has 1-12 carbons, may be straight chain, branched chain, and cyclic groups and with no more than 3 double bonds, all of which may be optionally substituted similarly to the alkyl group. Representative examples of lower alkenyl radicals in carboxylic acids include vinyl (ethenyl), allyl (propen-3-yl), 1-buten-4-yl; 2-buten-4-yl, 1-penten-5-yl, and the like.

The term “pharmaceutically-acceptable carboxylic acid” means a carboxylic acid moiety that is useful for forming the pharmaceutical formulations and compositions are also physiologically acceptable and generally non-toxic to a subject receiving the moiety.

Pharmaceutical Compositions

Methods of nasal administration of hormone-based drugs are known, for example, an oil based vehicle for drug administration are described in U.S. Patent Publication No. US 2012-0009250 A1 and U.S. Patent Publication No. 2017-0281644 A1, which are incorporated herein by reference in their entireties.

Some examples of lipid-based vehicles for oral delivery have been described in U.S. Pat. No. 6,096,338, which is incorporated herein by reference in its entirety. Silica similar to this has been used to make thixotropic compositions since the 1980's and is described in U.S. Pat. No. 4,497,918, which is incorporated herein by reference in its entirety. Thixotropic macro-emulsions comprising both oil and aqueous phases useful in the invention are described in U.S. Patent U.S. Patent Publication No. 2017-0348276 A1, which is incorporated herein by reference in its entirety.

According to certain embodiments, the formulation comprises: (1) an esterified testosterone derivative; (2) an oily vehicle; and (3) a wetting agent or mixture of wetting agents and/or a pharmaceutically acceptable surfactant or mixture of surfactants.

According to other embodiments, the formulation comprises: (1) an esterified testosterone derivative; (2) an oily vehicle; (3) a wetting agent or mixture of wetting agents and/or a pharmaceutically acceptable surfactant or mixture of surfactants; and (4) a thickening agent.

According to other embodiments, the formulation comprises: (1) an esterified testosterone derivative; (2) an oily vehicle; (3) a wetting agent or mixture of wetting agents and/or a pharmaceutically acceptable surfactant or mixture of surfactants, (4) a thickening agent; and (5) optionally water.

According to other embodiments, the formulation comprises: (1) an esterified testosterone derivative; (2) an oily vehicle; (3) a wetting agent or mixture of wetting agents and/or a pharmaceutically acceptable surfactant or mixture of surfactants; (4) a thickening agent; and (5) optionally water;, wherein the combination or mixture of ingredients leads to a thixotropic mixture.

According to other embodiments, the formulation comprises: (1) an esterified testosterone derivative; (2) an oily vehicle; (3) a wetting agent or mixture of wetting agents and/or a pharmaceutically acceptable surfactant or mixture of surfactants and (4) a thickening agent, such as colloidal silica; wherein the combination of ingredients leads to a thixotropic mixture.

According to certain embodiments, the oily vehicle is selected from the group consisting of: a pharmaceutically acceptable vegetable oil, a monoglyceride, a diglyceride, Sucrose acetate isobutyrate (SAIB), a synthetic triglyceride, a synthetic oil and any combination or mixture thereof.

According to certain embodiments, the pharmaceutically acceptable vegetable oil is selected from the group consisting of: Almond Oil Sweet (Prunus dulcis), Almond Oil Virgin (Prunus amygdalus), Aloe Vera Oil (Aloe barbadensis), Apricot Kernel Oil (Prunus armeniaca), Argan Oil (Argania spinosa), Avocada Oil (Persea americana), Apricot Oil (Prunus armeniaca), Amla Oil (Emblica officinalis), Borage Oil (Borago officinalis), Black Seed Oil (Nigella sativa), Castor Oil (Ricinus communis), Carrot Oil (Daucus carota), Coconut Oil (Cocus nucifera), Corn Oil, Cucumber Oil (Cucumis sativa), Chaulmogra Oil (Hydnocarpus wightianus), Emu Oil (Dromaius novae-Hollandiae), Evening Primrose Oil (Oenothera biennis), Flaxseed Oil (Linum usitatissimum), Grapeseed Oil (Vitus vinifera), Hazel Nut Oil (Avekkana), Jojoba Oil Refined (Simmondsia chinensis), Moringa Oil (Moringa oliefera), Marula Oils (Sclerocarya birrea), Wheatgerm Oil, Triticum vulgare, Macadamia Oil, (Macadamia ternifolia), Musk Melon Oil (Cuvumis melon), Musk Oil (Abelmoschus moschatus), Mustered Oil, Neem Oil (Azadirachta indica), Olive Oil (Olea europaea), Peach Kernel Oil (Prunus persica), Peanut Oil (Arachis hypogeae), Pomegranate Oil, Punica granatum, Psoralea Oil (Psoralea corylifolia), Primrose Oil (Oenothera bienni), Papaya Seed Oil (Carica papaya), Rosehip Seed Oil (Rosa rubiginosa), Safflower Oil, Seasame Seed (Refined) (Sesamum indicum), Sea Buckthorn Oil (Hippophae rhamnoides), Soya Bean Oil (Soja hispida), Sunflower Oil (Helianthus annus), Sweet Almond Oil (Prunus amygdalus Var. Dulcus), Sweet Cherry Kernel Oil (Prunus avium), Walnut Oil (Juglans regia), Water Melon Oil (Citrullus vulgaris).

Pharmaceutically acceptable synthetic oils according to the invention include SAI B, polyethylene glycol (PEG), polyethyleneglycol-polypropylene glycol (poloxamers), alkyl-modified PEG or poloxamers, silicone and mineral oil

According to certain preferred embodiments, the oily vehicle comprises medium chain triglycerides, castor oil, sesame oil, PEG, Poloxamer, SAIB or mixtures thereof.

According to certain embodiments, the testosterone therapeutic active, or mixture of actives, is selected from one or more of the group consisting of the compounds described by Formula 1 above and as described in Examples 1 and 2.

According to certain embodiments, the testosterone therapeutic active is preferably selected from the group consisting of the compounds described testosterone proprionate, testosterone enanthate, testosterone cypionate, testosterone undecanoate, and combinations or mixtures thereof.

According to certain embodiments, the a wetting agent or mixture of wetting agents and/or a pharmaceutically acceptable surfactant or mixture of surfactants is selected from the group consisting of: a polysorbate, a polyoxyethylene hydrogenated vegetable oil, a polyoxyethylene vegetable oil; a polyoxyethylene sorbitan fatty acid ester; a polyoxyethylene-polyoxypropylene block copolymer; a polyglycerol fatty acid ester; a polyoxyethylene glyceride; a polyoxyethylene sterol, or a derivative or analogue thereof; a reaction mixture of polyols and at least one member of the group consisting of fatty acids, glycerides, vegetable oils, hydrogenated vegetable oils, fractionated oils and sterols; a tocopheryl polyethylene glycol succinate; a sugar ester; a sugar ether; a sucroglyceride; an alkylglucoside; an alkylmaltoside; an alkylthioglucosides; a lauryl macrogolglyceride; a polyoxyethylene alkyl ether; a polyoxyethylene alkylphenol; a polyethylene glycol fatty acid ester; a polyethylene glycol glycerol fatty acid ester; a polyoxyethylene sorbitan fatty acid ester; a polyoxyethylene-polyoxypropylene block copolymer such as poloxamer-108, 188, 217, 238, 288, 338, 407, 124, 182, 183, 212, 331, or 335, and combinations or mixtures thereof; an ionic hydrophilic surfactant such as sodium dodecyl sulphate or docusate sodium; a bile acid; a cholic acid; a deoxycholic acid; a chenodeoxycholic acid; salts thereof, and combinations or mixtures thereof.

According to certain embodiments, the formulation further comprises a rheology modifying (thickening agent) agent. The thickening agent would preferably be added to the majority liquid phase (oil or water) of the formulation. For formulations where the majority phase is an oil (with or without an aqueous phase), the pharmaceutically acceptable thickening agents would be selected from colloidal silica, silicates, alumina, a high molecular weight polymer or a solid/waxy substance, bee wax, alumina, silica, colloidal silica, silicates and high melting waxes, cetostearyl alcohol and combinations or mixtures thereof. For formulations where the majority phase is aqueous, the thickener would be a pharmaceutically acceptable hydrophilic polymer selected from the group consisting of: HPMC, HPC, Sodium CMC, Sodium CMC and MCC, natural gums like Xanthan gum, Guar gum, gum acacia, gum tragacanth, starches like maize starch, potato starch, pregelatinized starch and combinations or mixtures thereof. Thickening agents may be added to both phases in a mixed phase system.

According to certain embodiments, a formulation comprising water may further comprise, a surfactant and an osmotic complement.

According to certain embodiments, the surfactant is selected from the group consisting of: Glycol Distearate, Sorbitan Trioleate, Propylene Glycol Isostearate, Glycol Stearate, Sorbitan Sesquioleate, Lecithin, Sorbitan Oleate, Sorbitan Monostearate NF, Sorbitan Stearate, Sorbitan Isostearate, Steareth-2, Oleth-2, Glyceryl Laurate, Ceteth-2, PEG-30 Dipolyhydroxystearate, Glyceryl Stearate SE, Sorbitan Stearate (and) Sucrose Cocoate, PEG-4 Dilaurate, Methyl Glucose Sesquistearate, Lecithin HLB (variable) PEG-8 Dioleate, Sorbitan Laurate, Sorbitan Laurate , PEG-40 Sorbitan Peroleate, Labrafil M1944CS, Laureth-4, PEG-7 Glyceryl Cocoate, PEG-20 Almond Glycerides, PEG-25 Hydrogenated Castor Oil, Stearamide MEA, Glyceryl Stearate (and) PEG-100 Stearate, Polysorbate 85, PEG-7 Olivate, Cetearyl Glucoside, Stearamide MEA , PEG-8 Oleate, Polyglyceryl-3 Methyglucose Distearate, Oleth-10, Oleth-10/Polyoxyl 10 Oleyl Ether NF, Ceteth-10, PEG-8 Laurate, Cocamide MEA, Polysorbate 60 NF, Polysorbate 60, Polysorbate 80, Isosteareth-20, PEG-60 Almond Glycerides, PEG-20 Methyl Glucose Sesquistearate, Ceteareth-20, Oleth-20, Steareth-20, Steareth-20 , Steareth-21, Steareth-21 , Ceteth-20, Steareth-100 and combinations or mixtures thereof.

According to certain preferred embodiments, the testosterone therapeutic active is a propionate ester of the active testosterone, the oily vehicle is castor oil, and the wetting agent is oleoyl polyoxylglycerides. Optionally, silica is used as the preferred thickener.

Compounds useful in this invention are those of formula (I), as defined herein. Active ester derivatives of testosterone (3a-hydroxy-3b-methyl-5a-prenan-20-one) are preferred compounds. As mentioned hereinbefore, a physiologically cleavable ester of the 3-hydroxy group, especially of testosterone, is also useful. While the carboxylic acids from which such esters may be derived were generically mentioned previously, the following is a list of carboxylic acids useful to form the esters at the 3-position: acetic acid, n-propionic acid, n-butyric acid, t-butyl carboxylic acid, n-pentanoic acid, benzoic acid, heptanoic acid, cyclopentylpropionic acid, undecanoic acid, morpholinocarboxylic acid, malonic acid, succinic acid, glutaric acid, adipic acid, pimelic acid, suberic acid, n-propenoic acid, e-butenoic acid, and the like. .

Compositions of the invention may be administered by any suitable route which will introduce the intended active ester derivative(s) of testosterone to the patient in a soluble form and therefore overcome the solubility limitation of testosterone. The compositions of the invention are prodrugs and after administration, via the action of hydrolases or natural hydrolysis, are converted to testosterone. The mode of administration may be pernasal or intranasally.

Pharmaceutically acceptable excipients include such as solvents, diluents, binders, lubricants, preservatives, disintegrants, wetting agents, surfactants, stabilizers, anti-oxidants, coloring agents, flavors, sweetners, and the like. Examples of these excipients can be found in the standard publication Remington's Pharmaceutical Sciences, 19 Edition, Mack Publishing Co., Easton, Pa.-1995 (“Remington's”), which is incorporated herein by reference in its entirety. Person skilled in the art will recognize that certain substances can equally act as surfactant agents in one formulation and wetting agents in another. According to the invention, it is noted that any agent in either of these categories can be used interchangeably in the other category depending on the nature of the formulation.

Dosage forms according to the invention include liquids, oils, emulsions, semi-solid emulsions, suspensions, gels, creams, ointments, solids, waxes, etc., which can be administered intranasally to a patient. The preferred dosage form is one that provides the drug to the patient in an efficient and convenient manner while achieving the safety and efficacy for the desired condition.

The preferred dosage of a chosen drug will depend upon both the potency of the drug, the status of the patient and the nature of the condition to be treated. The composition will need to be prescribed by a treating physician, who will take into account any relevant factors, such as the age and weight of the patient, the severity of the patient's symptoms, the treatment regimen, and the chosen pernasal route of administration.

Depending on the dosage form and the administration route, the amount of the active compound in the composition to be administered will be sufficient to deliver the desired amount of active to the subject being treated to alleviate the medical condition, i.e., a therapeutically effective amount. Thus another aspect of the invention is the use of a component of formula (I) to prepare a composition useful for the treatment of a medical condition. The compound is confined with an excipient to form an acceptable formulation then combined with a label providing instructions for administration.

Another aspect of this invention is a pharmaceutical composition suitable for treating a medical condition, which composition comprises a compound of formula (I) and a pharmaceutically-acceptable excipient. Generally, the amount of the active compound will vary from about 1 milligram (mg) to about 135 mg per dosage unit, preferably about 2 mg-50 mg, and most preferably about 5 mg-35 mg. Depending on the size of the dosage form, the active may vary between about 1% to about 90% by weight, preferably less than 50% by weight.

Thus the percentage of the active may be, e.g., 1, 2, 3, 4, 5, 10, 20, 30, 40, 50 percent or any intermediate percentage or range as desired. By using a dosage form with the desired composition percentage, a doctor skilled in the art can administer enough to achieve about 0.1 mg/kilogram (kg) body weight in the subject to about 100 mg/kg, preferably about 0.1 mg/kg to about 10 mg/kg. The label that accompanies the dosage form will provide instructions for using the composition to treat the medical condition. Treatment can be on an as-needed, acute, subchronic (for a short period of time) or on a chronic basis.

Compositions according to the invention may be used to treat a number of conditions. The preferred dose and route of administration may depend on the nature of the condition to be treated. Conditions that may be treated with testosterone esters according to the invention may include without limitation: hypogonadism, anxiety, fear, sexual dysfunction, cancer, declining sexual drive, libido, testosterone replacement therapy, low testosterone, depression, anemia, prostate cancer and breast cancer.

The following examples are provided as a guide for a practitioner of ordinary skill in the art. The examples should not be construed as limiting the invention, but as examples merely providing exemplary methodology useful in understanding and practicing the invention.

### EXAMPLES

### Example 1

About 14% Testosterone Cypionate in a Castor Oil Formulation

| Ingredient | % w/w |
|---|---|
| Testosterone cypionate | 14.00 |
| Castor oil | 82.00 |
| Oleoyl polyoxylglycerides | 4.00 |

Castor oil (about 82 parts) and oleoyl polyoxylglycerides (about 4 parts) are mixed together thoroughly. Testosterone cypionate (about 14 parts) is added and is dissolved upon mixing to form a clear gel or a viscous solution. The equivalent concentration of testosterone active is about 9.8%. A single dose using 125 μL pump (one actuation in each nostril), it will provide a total dose of about 24 mg testosterone equivalent or a dose of about 12 mg testosterone equivalent per nostril.

### Example 2

About 15% Testosterone Propionate in a Castor Oil Formulation

| Ingredient | % w/w |
|---|---|
| Testosterone propionate | 15.00 |
| Castor oil | 81.00 |
| Oleoyl polyoxylglycerides | 4.00 |

Castor oil (81 parts) and oleoyl polyoxylglycerides (about 4 parts) are mixed together thoroughly. Testosterone propionate (about 15 parts) is added and is dissolved upon mixing to form a clear gel or a viscous solution. The equivalent concentration of testosterone active is about 12.9%.

### Example 3

About 30% Testosterone Enanthate in a Castor Oil Formulation

| Ingredient | % w/w |
|---|---|
| Testosterone enanthate | 30.00 |
| Castor oil | 66.00 |
| Oleoyl polyoxylglycerides | 4.00 |

Castor oil (about 66 parts) and oleoyl polyoxylglycerides (about 4 parts) is mixed together thoroughly. Testosterone enanthate (about 30 parts) is added and is dissolved upon mixing to form a clear gel or a viscous solution. The equivalent concentration of testosterone active is about 22.5%.

### Example 4

About 36% Testosterone Enanthate in a Castor Oil Formulation

| Ingredient | % w/w |
|---|---|
| Testosterone enanthate | 36.3 |
| Castor oil | 60.00 |
| Oleoyl polyoxylglycerides | 3.7 |

Castor oil (about 60 parts) and oleoyl polyoxylglycerides (about 3.7 parts) is mixed together thoroughly. Testosterone enanthate (about 36.3 parts) is added and is dissolved upon mixing to form a clear gel or a viscous solution. The equivalent concentration of testosterone active is about 26.1%.

### Example 5

About36% Testosterone Enanthate in a Castor Oil Formulation

| Ingredient | % w/w |
|---|---|
| Testosterone enanthate | 36.30 |
| Castor oil | 58.60 |
| Oleoyl polyoxylglycerides | 2.55 |
| Colloidal silicon dioxide | 2.55 |

Colloidal silicon dioxide (about 4 parts) was dispersed in castor oil (about 92 parts). Added oleoyl polyoxylglycerides (about 4 parts) and is mixed to form a uniform gel. Warmed the about 63.7 parts of this mixture to about 40° C. and dissolve testosterone enanthate (about 36.3 parts) to form a clear gel. The equivalent concentration of testosterone active is about 26.1%.

### Example 6

Testosterone Ester Mixture in Castor Oil Formulation

| Ingredient | % w/w |
|---|---|
| Testosterone cypionate | 4.90 |
| Testosterone propionate | 4.90 |
| Testosterone enanthate | 20.00 |
| Castor oil | 62.2 |
| Oleoyl polyoxylglycerides | 4.0 |
| Colloidal silicon dioxide | 4.0 |

Colloidal silicon dioxide (about 4 parts) is dispersed in castor oil (about 62.2 parts). The mixture is warmed to about 40° C. and testosterone cypionate (about 4.9 parts), testosterone propionate (about 4.9 parts) and testosterone enanthate (about 20 parts) are added and mixed to form a clear solution. Oleoyl polyoxylglycerides (about 4 parts) is added and is mixed to produce a uniform gel. The equivalent concentration of testosterone active is about 21.9%.

### Example 7

About 36% Testosterone Enanthate Formulation

| Ingredient | % w/w |
|---|---|
| Testosterone enanthate | 36.30 |
| Castor oil | 55.70 |
| Oleoyl polyoxylglycerides | 4.0 |
| Colloidal silicon dioxide | 4.0 |

Colloidal silicon dioxide (about 4 parts) is dispersed in medium chain triglycerides (about 55.7 parts). The mixture is warmed to about 40° C. and then testosterone enanthate (about 36.3 parts) is dissolved to form a clear mixture. Oleoyl polyoxylglycerides (about 4 parts) is then added and is mixed to form a uniform clear gel. The equivalent concentration of testosterone active is about 26.1%.

### Example 8

About 36% Testosterone Enanthate Formulation

| Ingredient | % w/w |
|---|---|
| Testosterone enanthate | 36.30 |
| Sesame oil | 55.70 |
| Oleoyl polyoxylglycerides | 4.0 |
| Colloidal silicon dioxide | 4.0 |

Colloidal silicon dioxide (about 4 parts) is dispersed in sesame oil (about 55.7 parts). The mixture is warmed to about 40° C. and the testosterone enanthate (about 36.3 parts) is added to dissolve and form a clear mixture. Oleoyl polyoxylglycerides (about 4 parts) is added and is mixed to form a uniform clear gel. The equivalent concentration of testosterone active is about 26.1%.

### Example 9

About 36% Testosterone Enanthate Formulation

| Ingredient | % w/w |
|---|---|
| Testosterone enanthate | 36.30 |
| Castor oil | 20.00 |
| Sesame oil | 15.70 |
| Medium chain triglycerides | 20.00 |
| Oleoyl polyoxylglycerides | 4.00 |
| Colloidal silicon dioxide | 4.00 |

Colloidal silicon dioxide (about 4 parts) is dispersed into a mixture of sesame oil (about 15.7 parts), medium chain triglycerides (about 20 parts) and castor oil (about 20 parts). The mixture is warmed to about 40° C. and then testosterone enanthate (about 36.3 parts) is added to dissolve and form a clear mixture. Oleoyl polyoxylglycerides (about 4 parts) is added and is mixed to form a uniform clear gel. The equivalent concentration of testosterone active is about 26.1%.

### Example 10

Testosterone Ester Mixture Formulation

| Ingredient | % w/w |
|---|---|
| Testosterone cypionate | 4.90 |
| Testosterone propionate | 4.90 |
| Testosterone enanthate | 20.00 |
| Sesame oil | 15.70 |
| Medium chain triglycerides | 20.00 |
| Castor oil | 62.2 |
| Oleoyl polyoxylglycerides | 4.0 |
| Colloidal silicon dioxide | 4.0 |

Colloidal silicon dioxide (about 4 parts) is dispersed into a mixture of sesame oil (20 parts), medium chain triglycerides (21.2 parts) and castor oil (about 21 parts). The mixture is warmed to about 40° C. and then testosterone enanthate (about 20 parts), testosterone propionate (about 4.9 parts) and testosterone cypionate (about 4.9 parts) are dissolved to form a clear mixture. Oleoyl polyoxylglycerides (about 4 parts) is added and is mixed to form a uniform clear gel. The equivalent concentration of testosterone active is about 21.9%.

### Example 11

Emulsion Formulation of Testosterone Esters

| Ingredient | % w/w |
|---|---|
| Testosterone enanthate | 36.30 |
| Medium chain triglycerides | 30.00 |
| Polyoxyl 35 castor oil | 2.00 |
| Carbomer 971P | 0.40 |
| Purified water | 31.30 |

Medium chain triglycerides (about 30 parts) and polyoxyl 35 castor oil (about 2 parts) are mixed. The mixture is heated to about 60° C. and then testosterone enanthate (about 36.3 parts) is added and is dissolved to form a clear oily solution. Separately, Carbomer 971P (about 0.4 parts) is dispersed in water (about 31.3 parts) at about 65° C. The oil phase-drug solution is added to the water phase-solution and is emulsified. The pH is adjusted with about 1N NaOH solution to about 6.5 to about 7.5. The mixture is cooled to room temperature with continuous mixing to form a white opaque aqueous gel. The equivalent concentration of testosterone active is about 26.1%.

### Example 12

Nasal Dispenser Containing Testosterone

| Ingredient | % w/w |
|---|---|
| Testosterone propionate | 0.80 |
| Castor oil | 91.20 |
| Oleoyl polyoxylglycerides | 4.00 |
| Colloidal silicon dioxide | 4.00 |

Testosterone propionate (0.80 parts) is dissolved in castor oil (91.80 parts). Colloidal silicon dioxide (4 parts) is dispersed in it. Oleoyl polyoxylglycerides (4 parts) is added and mixed to form a uniform gel. The equivalent concentration of testosterone active is about 0.67%. When used with a 75 μL pump, this will provide a dose of about 0.5 mg per actuation or a total dose of about 1 mg when used in both nostrils.

### Example 13

Testosterone Enanthate for Female Sexual Dysfunction

| Ingredient | % w/w |
|---|---|
| Testosterone enanthate | 1.85 |
| Castor oil | 92.15 |
| Oleoyl polyoxylglycerides | 4.00 |
| Beeswax | 2.00 |

Beeswax (2 parts) is dissolved in castor oil (92.15 parts) at about 60° C. Oleoyl polyoxylglycerides (4 parts) is added and mixed well. Testosterone enanthate (1.85 parts) is dissolved to form a uniform gel. The equivalent concentration of testosterone active is about 1.4%. When used with a 75 μL pump (one actuation in each nostril), this will provide a total dose of about 2 mg testosterone equivalent or about 1 mg testosterone equivalent per nostril.

### Example 14

Testosterone Cypionate for Female Sexual Dysfunction

| Ingredient | % w/w |
|---|---|
| Testosterone cypionate | 4.77 |
| Coconut oil | 93.23 |
| Beeswax | 2.00 |

Beeswax (2 parts) is mixed with castor oil (93.23 parts) at about 60° C. Oleoyl polyoxylglycerides (4 parts) is added and mix well. Testosterone cypionate (4.77 parts) is dissolved to form a uniform gel. The equivalent concentration of testosterone active is about 3.34%. When used with a 75 μL pump (one actuation in each nostril), this will provide a total dose of about 5 mg testosterone equivalent or about 2.5 mg testosterone equivalent per nostril.

### Example 15

Testosterone Propionate Gel for Female Sexual Dysfunctions

| Ingredient | % w/w |
|---|---|
| Testosterone propionate | 4.00 |
| Medium chain triglycerides | 30.00 |
| Castor Oil | 30.00 |
| Polyoxyl 35 castor oil | 2.00 |
| Carbomer 971P | 0.40 |
| Purified water | 33.60 |
| Sodium hydroxide Solution 1N | q.s. for pH 7.0 |

Medium chain triglycerides (30 parts), castor oil (30 parts) and polyoxyl 35 castor oil (2 parts) are mixed. The mixture is heated to about 60° C. and then testosterone propionate (4 parts) is added and dissolved to form a clear oily solution. Separately, carbomer 971P (0.4 parts) is dispersed in water (31.3 parts) at about 65° C. The oil phase-drug solution is added to the aqueous phase and emulsified. The pH is adjusted with about 1N NaOH solution to about pH 6.5 to 7.5. The mixture is cooled to room temperature with continuous mixing to form a white opaque aqueous gel. The equivalent concentration of testosterone active is about 3.36%. When used with a 75 μL pump in both nostrils, it provides a total dose of about 5 mg testosterone equivalent or about 2.5 mg testosterone equivalent per nostril.

### Example 16

Testosterone Enanthate for Treatment of Prostate Cancer

| Ingredient | % w/w |
|---|---|
| Testosterone enanthate | 18.50 |
| Castor oil | 73.50 |
| Oleoyl polyoxylglycerides | 4.0 |
| Colloidal silicon dioxide | 4.0 |

Colloidal silicon dioxide (4 parts) is dispersed in castor oil (73.00 parts). The mixture is warmed to about 40° C. and then testosterone enanthate (19.00 parts) is dissolved to form a clear mixture. Oleoyl polyoxylglycerides (4 parts) is then added and mixed to form a uniform clear gel. The equivalent concentration of testosterone active is about 13.67%. When used with a 125μL pump (one actuation in each nostril), it will provide a total dose of about 33.5 mg testosterone equivalent or about 16.75 mg testosterone equivalent per nostril.

### Example 17

Testosterone Propionate for Prostate Cancer

| Ingredient | % w/w |
|---|---|
| Testosterone propionate | 10.50 |
| Castor oil | 81.50 |
| Oleoyl polyoxylglycerides | 4.0 |
| Colloidal silicon dioxide | 4.0 |

Colloidal silicon dioxide (4 parts) is dispersed in castor oil (81 parts). The mixture is warmed to about 40° C. and then testosterone propionate (11 parts) is dissolved to form a clear mixture. Oleoyl polyoxylglycerides (4 parts) is then added and mixed to form a uniform clear gel. The equivalent concentration of testosterone active is about 9.2%. When used with a 125 μL pump (one actuation in each nostril), it will provide a total dose of about 22.6 mg testosterone equivalent or about 11.3 mg testosterone equivalent per nostril.

### Example 18

Testosterone Cypionate for Prostate Cancer

| Ingredient | % w/w |
|---|---|
| Testosterone cypionate | 6.50 |
| Castor oil | 85.50 |
| Oleoyl polyoxylglycerides | 4.0 |
| Colloidal silicon dioxide | 4.0 |

Colloidal silicon dioxide (4 parts) is dispersed in castor oil (85.5 parts). The mixture is warmed to about 40° C. and then testosterone propionate (6.5 parts) is dissolved to form a clear mixture. Oleoyl polyoxylglycerides (4 parts) is then added and mixed to form a uniform clear gel. The equivalent concentration of testosterone active is about 9.2%. When used with a 125 μL pump (one actuation in each nostril), it will provide a total dose of about 11 mg testosterone equivalent or about 5.5 mg testosterone equivalent per nostril.

### Example 19

Mixture of Testosterone Esters for Prostate Cancer

| Ingredient | % w/w |
|---|---|
| Testosterone cypionate | 6.00 |
| Testosterone enanthate | 6.00 |
| Testosterone propionate | 6.00 |
| Castor oil | 80.00 |
| Oleoyl polyoxylglycerides | 4.0 |
| Colloidal silicon dioxide | 4.0 |

Colloidal silicon dioxide (4 parts) is dispersed in castor oil (80 parts). The mixture is warmed to about 40° C. and then testosterone propionate, testosterone cypionate and testosterone enanthate (6 parts each) are dissolved to form a clear mixture. Oleoyl polyoxylglycerides (4 parts) is then added and mixed to form a uniform clear gel. The equivalent concentration of testosterone active is about 13.2%. When used with a 125 μL pump (one actuation in each nostril), it will provide a total dose of about 33 mg testosterone equivalent or about 16.5 mg testosterone equivalent per nostril.

### Example 20

Nasal Dispenser Containing Testosterone

The composition from Example 7 (36.3% testosterone enanthate ester) is filled into a nasal dispenser as described in U.S. Patent Publication No. 2017-0348276 A1 and then capped and sealed. The nasal dispenser provides 125 uL doses at each actuation. Use in the nose, this pharmaceutical dosage form can provide about 44.5 mg of testosterone ester per actuation (equivalent to a dose of about 32 mg testosterone) when administered to one nostril. Applied in both nostril, the total dose is about 89 mg of testosterone esters (equivalent to a total dose of about 64 mg testosterone).

### Example 21

Pharmacokinetics

A composition per Example 12 is prepared and comprises an about 9% testosterone propionate formulation in a mixture of castor oil, silica and oleoyl polyoxylglycerides contained in a nasal dispenser which delivers 125 uL per actuation, such that with each actuation delivers about 11 mg testosterone proprionate (equivalent to about 9.3 mg testosterone). The composition is administered as a single dose, one actuation applied in each nostril, to a hypogonadal patient to achieve a total dose of about 22mg testosterone proprionate (equivalent to about 18.6 mg testosterone). Blood samples are obtained prior to the dose and then at about 20, about 40, about 60, about 80 and about 100 min after the dose, followed by additional blood samples at about 2 h, about 4 h, about 8 h, about 12 h and about 24 h.

All publications, including any and all patents, patent applications, U.S. patent publications and PCT publications, referencedherein are incorporated here i n by reference in their entireties as if each has been fully set forth herein.

Various modifications and variations of the described method and system of the invention will be apparent to those skilled in the art without departing from the scope and spirit of the invention. Although the invention has been described in connection with specific exemplary preferred embodiments, it should be understood that the invention as claimed should not be unduly limited to such specific exemplary embodiments. Indeed, various modifications of the described modes for carrying out the invention are intended to be within the scope of the following claims.

Having described our invention, we claim:

1. A pharmaceutical composition for nasal administration comprising (a) an effective amount of an active ester derivative of testosterone or a mixture of active ester derivatives of testosterone, and (b) a pharmaceutically acceptable oily vehicle.

2. The composition claim 1, wherein the pharmaceutically acceptable oily vehicle selected from any one or mixture of a vegetable oils and sucrose acetate isobutyrate (SAIB).

3. The composition of claim 1, wherein the pharmaceutically acceptable oily vehicle selected is a vegetable oil or mixture of vegetable oils.

4. The composition of claim 1, wherein the pharmaceutically acceptable oily vehicle is comprised of >75% castor oil.

5. The composition of claim 1, wherein the pharmaceutically acceptable oily vehicle is comprised of >75% medium chain triglycerides (MCT).

6. The composition of claim 1, wherein the pharmaceutically acceptable oily vehicle is SAIB.

7. A pharmaceutical composition of claim 4, wherein the active ester derivative of testosterone of claim 1 comprises about 1% to about 75% by weight of the pharmaceutical composition.

8. A pharmaceutical composition of claim 1, which further comprises a wetting agent.

9. A pharmaceutical composition of claim 1, which further comprises a thickener.

10. A pharmaceutical composition of claim 1, which further comprises water.

11. A pharmaceutical composition of claim 1, which is thixotropic.

12. A pharmaceutical composition of claim 1, which is a solid or wax.

13. A pharmaceutical composition of claim 1, wherein the composition is placed in a spray dispenser for administration to a subject in need thereof.

14. A pharmaceutical compositions of claim 1, wherein the pharmaceutical composition is a gel for administration to a subject in need thereof.

15. A pharmaceutical compostion of claim 1, wherein a single dose of said pharmaceutical composition delivers up to about 100 mg of testosterone per day.

16. A method of use wherein a pharmaceutical composition of claim 1 allows a patient to self-medicate.

17. A method of use wherein a pharmaceutical composition of claim 1 allows a patient to achieve in a single daily administration to both nostrils a quantity of testosterone sufficient to treat an indication from which a subject suffers.

18. A method of use wherein a pharmaceutical composition of claim 1, wherein the therapy is a testosterone replacement therapy.

19. A method of use wherein a pharmaceutical composition of claim 1 comprises an effective mount of an active ester derivative of testosterone for achieving supraphysiological levels of testosterone for use to treat conditions selected from a group consisting of hypogonadism, anxiety, fear, sexual dysfunction, cancer, declining sexual drive, libido, testosterone replacement therapy, low testosterone, depression, anemia, prostate cancer and breast cancer.

20. A pharmaceutical composition of claim 2, which further comprises a wetting agent, a thickner, water or any mixtures thereof.

21. A pharmaceutical composition of claim 3, which further comprises a wetting agent, a thickner, water or any mixtures thereof.

22. A pharmaceutical composition of claim 4, which further comprises a wetting agent.

23. A pharmaceutical composition of claim 5, which further comprises a wetting agent, a thickner, water or any mixtures thereof.

24. A pharmaceutical composition of claim 6, which further comprises a wetting agent, a thickner, water or any mixtures thereof.

25. A pharmaceutical composition of claim 7, which further comprises a wetting agent, a thickner, water or any mixtures thereof.

26. A pharmaceutical composition of claim 4, which further comprises a thickner.

27. A pharmaceutical composition of claim 3, which further comprises water.

28. A pharmaceutical composition of claim 4, which further comprises water.

29. A pharmaceutical composition of claim 4, which is thixotropic.

30. A pharmaceutical composition of claim 3, which is a solid or wax.

31. A pharmaceutical composition of claim 4, which is a solid or wax.

32. A pharmaceutical composition of claim 4, wherein the composition is placed in a spray dispenser for administration to a subject in need thereof.

33. A pharmaceutical composition of claim 4, wherein the pharmaceutical composition is a gel for administration to a subject in need thereof.

34. A method of use wherein a pharmaceutical composition of claim 4 allows a patient to self-medicate.

35. A method of use wherein a pharmaceutical composition of claim 4 allows a patient to achieve in a single daily administration to both nostrils a quantity of testosterone sufficient to treat an indication from which a subject suffers.

36. A method of use wherein a pharmaceutical composition of claim 3, wherein the therapy is a testosterone replacement therapy.

37. A method of use wherein a pharmaceutical composition of claim 4, wherein the therapy is a testosterone replacement therapy.

38. A method of use wherein a pharmaceutical composition of claim 3 comprises an effective mount of an active ester derivative of testosterone for achieving supraphysiological levels of testosterone for use to treat conditions selected from a group consisting of hypogonadism, anxiety, fear, sexual dysfunction, cancer, declining sexual drive, libido, testosterone replacement therapy, low testosterone, depression, anemia, prostate cancer and breast cancer.

39. A method of use wherein a pharmaceutical composition of claim 4 comprises an effective mount of an active ester derivative of testosterone for achieving supraphysiological levels of testosterone for use to treat conditions selected from a group consisting of hypogonadism, anxiety, fear, sexual dysfunction, cancer, declining sexual drive, libido, testosterone replacement therapy, low testosterone, depression, anemia, prostate cancer and breast cancer.
